(19) United States
(12) Patent Application Publication
(10) Pub. No.: US 2019/0083479 A1
(43) Pub. Date: Mar. 21, 2019

(54) COMBINATION MEDICATION

(71) Applicant: Keren Nehama Zaken BARASHI, Woodmead (ZA)

(72) Inventor: Keren Nehama Zaken BARASHI, Woodmead (ZA)

(21) Appl. No.: 15/710,440

(22) Filed: Sep. 20, 2017

(51) Int. Cl. A61K 31/455; A61K 31/191; A61K 31/194; A61K 31/28; A23L 33/00; C07D 213/80; A61K 9/20

(52) U.S. Cl. CPC A61K 31/455; A61K 31/191; A61K 31/194; A61K 2300/00; A23L 33/30; C07D 213/80; A61K 9/2077; A61K 31/28

(57) ABSTRACT

This invention provides a combination medication including an anti-diabetic prescription medication and at least one suitable complementary compound which is adapted to assist in increasing the effectiveness of the prescription medication and/or is adapted to assist in treating one or more symptoms of diabetes including moderating blood-sugar/glucose levels and/or reducing hunger or appetite levels.

### INTRODUCTION TO THE INVENTION

This invention relates to a combination medication. More particularly, this invention relates to a combination medication for diabetic patients.

### BACKGROUND TO THE INVENTION

Diabetes Mellitus, which is often simply referred to as diabetes, is a group of metabolic diseases in which a person has high blood-sugar/glucose. This occurs because the body does not produce sufficient insulin, or because cells do not respond to the insulin that is produced. The resulting high blood sugar produces various classical symptoms such as polyuria (frequent urination), polydipsia (increased thirst) and polyphagia (increased hunger).

The two main types of diabetes are Type 1 and Type 2 diabetes. Type 1 diabetes results from the body's failure to produce insulin, and at the present time requires insulin to be injected into a person. Type 2 diabetes results from insulin resistance, a condition in which cells fail to use insulin properly, sometimes combined with an insulin deficiency which can be controlled with hypoglycemic medication in the form of prescribed tablets such as a sulfonylurea, for example. All forms of diabetes have been treatable by means of insulin that first became available in 1921, and more recently by means of synthetic insulin. Both Type 1 and Type 2 diabetes are chronic conditions that usually cannot be cured.

Type 2 diabetes is the most common form of diabetes, and is often referred to as adult-onset diabetes. It is also associated with obesity.

Without proper treatment, diabetes can cause many complications such as hyperglycemia, coma, kidney failure and even blindness.

What is ideally required in treating Type 2 diabetes (although this also applies to Type 1 diabetes) is a multi-disciplinary or multi-facetted approach. This includes taking prescription medication, and should include controlling fat and cholesterol levels in the body, controlling weight, helping to regulate blood sugar levels, lowering serum triglycerides, and/or assisting a process in the body called thermogenesis.

Unfortunately, most diabetes patients are merely advised to watch or control their lifestyle and to avoid eating sweets or sweet foods or drinks. However, it appears that insufficient attention is paid in dealing with the abovementioned symptoms and problems experienced by diabetes patients.

It is understood that some diabetes patients take food or diet supplements but the disadvantage hereof is that these supplements, if used at all, are taken separately from anti-diabetic medication, and are often forgotten and/or taken too late to be effective. In addition, some weight control agents act on the central nervous system and hence can cause insomnia, nervousness, change in blood pressure or heart rate.

Accordingly, what is ideally required is a form of combination medication which assists the action of the anti-diabetic (prescription) medication and which assists in dealing with the aforementioned symptoms and problems, and which does not have the disadvantages associated with the prior art medications, as indicated above.

### OBJECTS OF THE INVENTION

Accordingly, it is an object of the present invention to provide a combination medication which overcomes, at least partly, the disadvantages associated with the prior art.

It is also an object of the present invention to provide a new and improved combination medication which involves an inventive step.

### SUMMARY OF THE INVENTION

According to a first aspect of the present invention, there is provided a combination medication including an anti-diabetic prescription medication and at least one suitable complementary compound which is adapted to assist in increasing the effectiveness of the prescription medication and/or is adapted to assist in treating one or more symptoms of diabetes including moderating blood-sugar/glucose levels and/or reducing hunger or appetite levels.

According to a second aspect of the present invention, there is provided a combination medication including an anti-diabetic prescription medication and at least one complementary compound selected from a suitable extract or a major phyto-constituent of a Garcinia Cambogia plant/tree, or synthetic hydoxycitric acid, or a chemical complex compound thereof (hereinafter referred to as “HCA” or as “the HCA compound group”, as applicable).

According to a third aspect of the present invention, there is provided a combination medication including an anti-diabetic prescription medication and at least one complementary compound selected from a suitable Glucose Tolerant Factor compound including chromium nicotinate, chromium dinicotinate, chromium polynicotinate, chromium dinicocysteinate, or a chemical complex compound thereof (hereinafter referred to as “GTF” or as “the GTF compound group”, as applicable).

According to a fourth aspect of the present invention, there is provided a combination medication including an anti-diabetic prescription medication and at least one suitable complementary compound selected from the HCA compound group and at least one suitable complementary compound selected from the GTF compound group.

The combination medication as described herein may be supplied in a single or combined tablet or capsule form, or when supplied as and in a separate tablet or capsule form optionally in a blister pack or blister cluster pack.

According to a fifth aspect of the present invention, there is provided a complementary compound adapted to assist in increasing the effectiveness of an anti-diabetes prescription medication and/or to assist in treating one or more symptoms of diabetes including moderating blood-sugar/glucose levels and reducing hunger or appetite levels, the complementary compound comprising at least one suitable compound selected from the HCA compound group and/or at least one suitable compound selected from the GTF compound group.

According to a sixth aspect of the present invention, there is provided a food supplement for diabetes patients that is adapted to assist in increasing the effectiveness of anti-diabetes medication and/or to assist in treating one or more symptoms of diabetes including moderating blood-sugar/glucose levels and reducing hunger or appetite levels, including at least one suitable compound selected from the HCA compound group and/or at least one suitable compound selected from the GTF compound group.

According to a seventh aspect of the present invention, there is provided a method of treating diabetes and diabetes patients, including the step of administering to a diabetes patient a combination medication, or a complementary compound, or a food supplement as herein described.

The anti-diabetic prescription medication may be a conventional medication such as sulfonylurea, metformin, or insulin, or another prescription medication that is already “off-patent” or in other words is no longer patent-protected. Although such prescription medication may include a trademarked anti-diabetic product that is taken by a diabetes sufferer, this invention does not include such products that are still patent-protected per se.

The combination medication may preferably be supplied in a single or combined tablet or capsule form/formulation or dosage. This may also be supplied optionally in the form of a single or as separate tablet, or a single capsule, or singly in a blister pack or cluster. This should ideally be taken by a diabetic patient approximately 30 minutes before a meal.

It will be understood that the dosage of the complementary compound(s) will be at a biologically or pharmacologically active level.

In the case of a Type 1 diabetic patient, the additional compounds, preferably in a combined form, should be taken by a patient at the time of the insulin injection.

The details and advantages of the aforementioned invention will be explained further hereinafter.

### DETAILED DESCRIPTION OF THE INVENTION

The invention will now be described in greater detail, by way of example, with reference to the following description.

A combination medication was prepared by combining one tablet of an anti-diabetic prescription medication (5 mg) in a capsule with about 250 mg of Garcinia Cambogia extract (60% HCA) and about 250 microgram of Chromium Polynicotinate (30 micro-gram of Cr). The aforementioned amounts of these substances are pharmacologically or biologically active amounts.

In provisional (private and confidential) tests carried out over a number of years, this combination medication has proved to provide an increased effectivity of the anti-diabetic prescription medication, and in in moderating blood glucose levels and in respect of appetite suppression.

This combination medication was taken about 30 minutes before a meal, in order to achieve optimal benefits from the combination medication.

In commercial practice, the aforementioned three constituents would be blended into a single tablet, or a single capsule, or singly in a blister pack or cluster pack, for example, in order to produce a combination medication in accordance with this invention.

The aforementioned quantities of the additional compounds may be varied, but must of course be kept at a pharmacologically or biologically active level. For example, from about 100 mg to about 800 mg of Garcinia Cambogia extract per dose may be used; and from about 10 microgram to about 250 microgram of Chromium Nicotinate or Chromium Polynicotinate per dose may be used.

In use, it appears that the mechanism or action of the HCA group compounds i.e. Hydroxycitric Acid or Garcinia Cambogia extract or major phyto constituent from this plant fills the glycogen stores in the liver and other tissues, thereby reducing appetite whilst increasing energy levels. It also appears to assist in lowering the production of Triglycerides and cholesterol and appears to increase thermogenesis namely the “burning” of calories.

Unlike chemical stimulants commonly used in weight loss products, these compounds do not act on the central nervous system. This means that these compounds do not cause insomnia, nervousness, change in blood pressure or heart rate, and their effectiveness is protracted.

Their main attributes are inter alia the following:
Assisting to reduce the body's ability to store fat;
lowering body mass through appetite control;
assisting in lowering serum triglycerides; and
assisting the thermogenesis process.

In respect of the GTF group compounds, it appears that the action or mechanism of chromium in certain forms is necessary for the formation of Glucose Tolerance Factor (GTF) which is the factor responsible for binding insulin to cell membrane receptors sites and which regulates the body's use of glucose to balance blood sugar levels. Chromium is also required in the metabolism and storage of fats, proteins and carbohydrates by the body. Chromium in certain forms is a mineral that humans need in trace amounts to help enhance the action of insulin. Persons who have Chromium deficiencies have been found to show conditions of diabetes and contain higher levels of cholesterol. The characteristics of the aforementioned Chromium III compounds include the following namely:
Regulating hunger;
Reducing hunger cravings;
Assisting to protect DNA;
Important for proper heart function;
Assisting to control fat and cholesterol levels in the body;
Important for protein metabolism;
Appears to assist in controlling body mass;
Assisting to moderate or regulate blood sugar (glucose).

However, chromium alone has virtually no effect on insulin in vitro. It appears to require the presence of Vitamin B3 i.e. niacin to bind with chromium. This combination strongly potentiates the action of insulin in vivo.

The combination medication described herein ie in a single tablet/capsule form has a dual activity. Firstly, it assists in improving the activity and effectiveness of the anti-diabetes prescription medication and secondly, it simultaneously serves to reduce and control, at least partly, the symptoms and conditions or complications associated with diabetes such as high cholesterol levels, increased body mass, unhealthy heart functions, and other complications as set out above.

Instead of, or in addition to chromium nicotinate or chromium polynicotinate, other compounds or chemical complexes such as a HCA complex thereof, or a chromium nicotinate—HCA complex, or a chromium dinicotinate—HCA complex, may be used, for example.

Referring to some of the more complex GTF group compounds, it appears that chromium dinicocysteinate provides improved physical properties and increased chromium bio-availability and efficacy when administered to a patient. Chromium dinicocysteinate is a trivalent Chromium complex with two nicotinic acid ligands and one cysteine ligand. This chemical complex and related complexes can be used to improve anti-diabetic conditions and related health conditions.

Further chromium complexes can also be used such as chromium dinicocysteinate-hydroxycitrate (HCA) complex which is a complex of a GTF group compound including chromium with one of the HCA group compounds. This provides a doubly beneficial effect namely through the HCA group compound and through the above bio-available GTF group compound (including chromium).

It appears that these particular compounds/complexes can offer additional benefits to patients such as treating inflammation; reducting oxidative stress; improving liver and/or kidney function; treating atherosclerosis; treating symptoms associated with cardiovascular disease; lowering LDL cholesterol and/or increasing HDL cholesterol levels; and lowering body mass, body fat and/or body mass index and/or increasing lean body mass.

The amount of the GTF group compound namely the chromium complex required to be administered to a patient is typically from about 10 micro grams to about 500 micrograms, or more particularly from about 20 micrograms to about 250 micrograms.

It will therefore be seen from the aforementioned that an effective combination medication is provided that not only assists a diabetes patient inter alia with appetite suppression, and hence longer term mass reduction, but also assists in making the anti-diabetic prescription medication more effective. The combination medication provides a convenient and effective means of assisting a diabetes patient not only with their central problem but also with surrounding symptoms/problems and conditions in order to make the prescribed anti-diabetic medication more effective. This assists in dealing with and improving their overall condition and health.

Although various examples and embodiments of the invention have been described above, it will be readily understood by any person skilled in this field that other modifications, variations and possibilities of the invention are possible. Such modifications, variations and possibilities are therefore to be considered as falling within the spirit and scope of the present invention as herein claimed or described and/or exemplified.

1. A combination medication including an anti-diabetic prescription medication and at least one suitable complementary compound which is adapted to assist in increasing the effectiveness of the prescription medication and/or is adapted to assist in treating one or more symptoms of diabetes including moderating blood-sugar/glucose levels and/or reducing hunger or appetite levels.

2. A combination medication including an anti-diabetic prescription medication and at least one suitable complementary compound selected from an extract or a major phyto-constituent of the Garcinia Cambogia plant/tree, or synthetic hydroxycitric acid, or a chemical complex compound thereof (hereinafter referred to as “HCA” or “the HCA compound group”, as applicable).

3. A combination medication including an anti-diabetic prescription medication and at least one suitable complementary compound selected from a Glucose Tolerant Factor compound including chromium nicotinate, chromium dinicotinate, chromium polynicotinate, chromium dinicocysteinate, or a chemical complex compound thereof (hereinafter referred to as “GTF” or “the GTF compound group”, as applicable).

4. A combination medication as claimed in claim 2, including at least one suitable complementary compound selected from the HCA compound group and at least one suitable complementary compound selected from the GTF compound group.

5. A combination medication as claimed in claim 1, including when supplied in a single or combined tablet or capsule form, or when supplied in a separate tablet or capsule form optionally in a blister pack or blister cluster pack.

6. A complementary compound adapted to assist in increasing the effectiveness of an anti-diabetes prescription medication and/or to assist in treating one or more symptoms of diabetes including moderating blood-sugar/glucose levels and reducing hunger or appetite levels, the complementary compound comprising at least one suitable compound selected from the HCA compound group and/or at least one suitable compound selected from the GTF compound group.

7. A food supplement for diabetes patients that is adapted to assist in increasing the effectiveness of anti-diabetes medication and/or to assist in treating one or more symptoms of diabetes including moderating blood-sugar/glucose levels and reducing hunger or appetite levels, including at least one suitable compound selected from the HCA compound group and/or at least one suitable compound selected from the GTF compound group.

8. A method of treating diabetes and diabetes patients, including the step of administering to a diabetes patient a combination medication as claimed in claim 1.

9. A method of treating diabetes and diabetes patients, including the step of administering to a diabetes patient a complementary compound as claimed in claim 6.

10. A method of treating diabetes and diabetes patients, including the step of administering to a diabetes patient a food supplement as claimed in claim 7.
